(19) United States
(12) Patent Application Publication
(10) Pub. No.: US 2010/0228106 A1
(43) Pub. Date: Sep. 9, 2010

(54) SYSTEM FOR MEASURING CHANGES IN SKIN PARAMETERS

(75) Inventor: Gabriel Khazaka, Cologne (DE)

Correspondence Address: MAIER & MAIER, PLLC, 1000 DUKE STREET, ALEXANDRIA, VA, 22314, US

(73) Assignee: Courage + Khazaka Electronic GmbH, Cologne (DE)

(21) Appl. No.: 12/715,635

(22) Filed: Mar. 2, 2010

(30) Foreign Application Priority Data
Mar. 3, 2009 (EP) 09 154 205.0

(51) Int. Cl. A61B 5/00

(52) U.S. Cl. 600/306

(57) ABSTRACT

In a system for measuring changes in skin parameters of living beings on a skin surface to be examined as a function of the residence time of a substance, comprising at least one measuring apparatus for measuring the skin parameters, it is provided that the measuring apparatus is coupled with at least one dispenser means which when operated dispenses a substance associated with this dispenser means and applicable on the skin surface to be examined, the measuring apparatus recording in a memory the time of actuation of the dispenser means and the time of the at least one measurement of the skin parameters for the detection of the residence time of the substance, as well as the skin parameters measured which are associated with the time of measurement.

### BACKGROUND OF THE INVENTION

1. Field of the Invention

The invention refers to a system and a method for measuring changes in skin parameters of living beings on a skin surface to be examined, in dependence on the effect of a substance, as defined in the precharacterizing part of claim 1 and claim 14, respectively.

2. Description of Related Art

From the European laid-open document EP-A-1 166 717, a device is known which is used in measuring skin parameters of living beings on a skin surface to be examined, wherein the elastic properties of a surface structure is measured with a probe arranged in a housing, a transmitter and at least one receiver.

Presently known systems for measuring skin parameters of living beings on a skin surface to be examined are often used in scientific studies which analyze the effectiveness of pharmaceutical or cosmetic preparations, especially skin creams. Such studies document changes in the skin as a function of the residence time of the substance. For this purpose, the skin parameters of the probands are detected after defined residence times of the skin cream. In present studies, the probands have to visit so-called “proband centres” for the skin measurement.

However, this entails the drawback that only probands can be examined that live in the vicinity of the “proband center”. Further, the examinations are rather time-consuming for the probands so that, for instance, working people are not available as probands.

### SUMMARY OF THE INVENTION

It is therefore an object of the present invention to provide a system and a method of the type described above, wherein changes in skin parameters of living beings can be determined in a simple manner as a function of the residence time of a substance applicable on the skin surface to be examined.

The object is achieved according to the invention with the features of claims 1 and 14.

The present invention advantageously provides that in a system for measuring changes in skin parameters of living beings on a skin surface to be examined as a function of the residence time of a substance, the measuring apparatus is coupled with a dispenser means which, upon operation, dispenses a substance associated with the dispenser means and applicable onto the skin surface to be examined, the measuring apparatus recording in a memory the time of actuation of the dispenser means and the time of the at least one measurement of the skin parameters for a determination of the residence time of the substance, as well as the skin parameters measured that are associated with the time of measurement.

This has the advantage that the measurements are stored as a function of the residence time. Thus, changes of the skin can be documented in a simple manner. Probands could take such a device home. In this manner, probands that do not live in the near vicinity of a “proband center” or which are working can participate in a study as well.

It is another advantage that the skin examinations can be made in shorter time intervals.

The substance may be a pharmaceutical or cosmetic preparation, especially a skin cream.

After a settable time interval or a plurality of settable time intervals, the measuring apparatus an issue a reminder signal, respectively. Thus, the probands can be given a certain schedule.

The measuring apparatus is adapted to identify the substance contained in the dispenser means by means of an identification code associated with a substance and to associate with this substance the measured values and the times of the measurements and the times of actuation of the dispenser means.

The measuring apparatus is adapted to transmit the measurement results and the times of the measurements and the times of actuation of the dispenser means, which are associated with a certain substance, to at least one external data receiver or transfer them to an exchangeable memory medium. Thus, the measurement results of probands that do not live in the near vicinity of a “proband centre” can be transmitted to the “proband center” in a simple manner.

The measuring apparatus may be equipped with a room climate sensor means.

The measuring apparatus is configured to detect the skin elasticity and/or the color of the skin (pigmentation of the skin) and/or the fat level of the skin and/or the sebum level of the skin and/or the pH value of the skin.

The measuring apparatus can comprise at least one sensor means with which the presence of substances to be applied on the skin surface can be checked by measurement techniques, the sensor means detecting a marker in a substance in order to determine whether the substance marked with the marker is present on the skin surface to be examined.

Preferably, the marker should not interact with the chemical components of the substance.

Preferably, the marker is not resorbable by the skin. The marker may consist of particles, the particles being of such a size that they cannot penetrate into the skin.

The marker may be a fluorescent substance and the sensor means can detect the marker by means of fluorescence measurement.

A method for measuring changes in skin parameters of living beings on a skin surface to be examined as a function of the residence time of a substance may include the following steps:
measuring the skin parameters on the untreated skin surface to be examined and storing the skin parameters measured,
dispensing a substance to be applied on the skin surface to be examined and applying the substance on the skin surface to be examined,
identifying the substance dispensed,
storing the time the substance was dispensed,
measuring again the skin parameters after one or a plurality of predetermined residence times, and
storing the time of the at least one new measurement of the skin parameters for a detection of the residence time of the substance.

The quantity of the substance dispensed can be measured and stored.

### BRIEF DESCRIPTION OF THE DRAWINGS

The following is a detailed description of an embodiment of the invention with reference to the drawings. The FIGURE schematically illustrates:

FIG. 1 a system for measuring changes in skin parameters o living beings according to the invention.

### DETAILED DESCRIPTION OF PREFERRED EMBODIMENTS

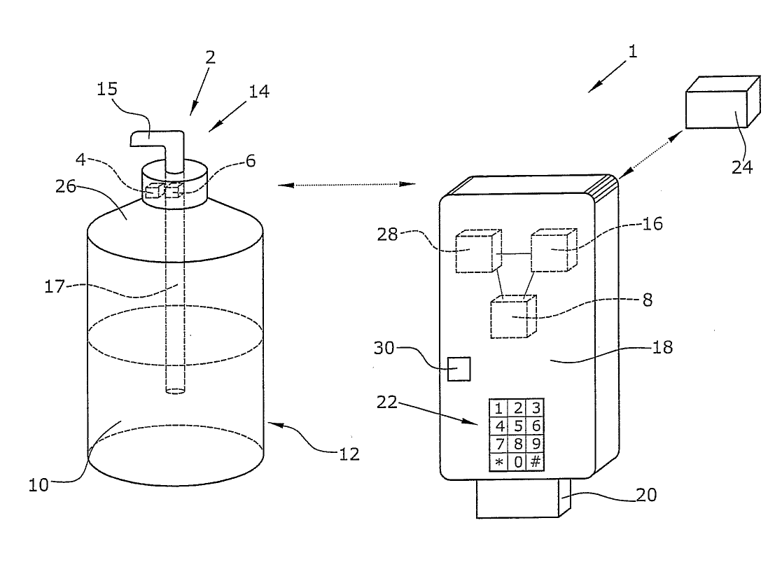

FIG. 1 illustrates a system for measuring changes in skin parameters of living beings. The system 1 comprises a dispenser means 2 and a measuring apparatus 18. The dispenser means 2 has a container 12 holding the substance 10. Preferably, the substance 10 is a skin cream. The container 12 can be closed with a lid 26. The lid 26 has an opening through which a tube 17 is guided that is part of a metering means 14. The top end of the tube 17 is provided with a metering head 15 formed integrally with the tube 17. The free end of the metering head 15 has an opening.

By actuation of the metering head 15 of the metering device 14, the substance 10 is pumped outward from the container 12 through the tube 17, the metering head 15 and the opening of the metering head onto the skin.

The dispenser means 2 further comprises a processor unit 4 and a memory unit 6. The processor unit 4 preferably comprises a clock for associating a time with data measured.

When the metering head 15 of the metering means 14 is actuated, the time of actuation is detected by the processor unit 4 and is stored in the memory unit 6. Likewise, the quantity of substance pumped outward is detected and stored in the memory unit 6. The time of actuation and the quantity of substance are stored in the memory unit 6 in an associated manner. For instance, the memory unit 6 stores that the quantity of substance b was dispensed at the time a.

The measuring apparatus 18 also comprises a processor unit 28 and a memory unit 16. The processor unit 28 also comprises a clock. Further, the measuring apparatus 18 includes a signal output 8, a keyboard 22 and a sensor means with sensors 20. The sensor means may, for instance, comprise sensors 20 for detecting the elasticity of the skin and/or the color of the skin and/or the fat level of the skin and/or the sebum level of the skin and/or the pH value of the skin and/or the skin temperature and/or the humidity of the skin.

The time at which the sensor unit measures the skin parameters is detected by the processor unit 28. Similarly, the processor unit 28 detects the associated measurement results measured during a measurement. The data are then supplied to the memory unit 16 where they are stored. The time of measurement and the associated measurement results are stored in the memory unit 16 in mutual association. Thus, it is stored in the memory unit 16 that at a time x the skin parameters y, z, w have been measured, for instance, wherein the skin parameters y, z, w may be the elasticity of the skin, the color of the skin and the pH value of the skin. Likewise, it can be determined how much time has elapsed since the actuation of the metering means 14 of the dispenser means 2. Since the application of the cream is approximately equal to the actuation of the metering means 14, the time elapsed since the actuation of the metering means corresponds to the residence time of the substance 10 or cream.

The measuring apparatus 18 may additionally comprise a room climate sensor means 30. The processor unit 4 of the dispenser means 2 and the processor unit 28 of the measuring apparatus 18 may communicate via a wireless local network. Thus, the data stored in the memory unit 6 can also be transmitted to the processor unit 28 from where they can be stored in the memory unit 16. Accordingly, it is also possible to use only one of the two memory units 6, 16.

Prior to the use of the system 1, the substance present in the dispenser means 2 is identified by means of an identification code associated with the substance 10. This identification may be effected, for instance, by entering the identification code into the measuring apparatus 18 via the keyboard 22. This identification code is then stored by the processor unit 28 in the memory unit 16 and/or is transmitted to the processor unit 4 where it is stored in the memory unit 6. Any data acquired thereafter by the processor unit 4 and/or the processor unit 28 are associated with this identification code and are stored in the memory unit 6 and/or the memory unit 16. Accordingly, the data stored in the memory unit 6 and/or the memory unit 16 can be associated with a substance 10.

In addition, a certain time interval can be set via the keyboard 22, which is stored in the memory unit 16 and/or the memory unit 6. When, after the actuation of the metering means 14, the time interval stored in the memory unit 16 and/or the memory unit 6 has lapsed, a signal is outputted by the signal output unit 8. The signal is to remind a proband to perform a skin measurement. It is also possible to input a plurality of time intervals and to store these in the memory unit 16 and/or the memory unit 6, wherein after the respective time intervals have lapsed, a measurement should be made using the measuring apparatus 18. Further, specific times of the day can be stored in the memory unit 16 and/or the memory unit 6, according to which a signal is outputted to remind a proband to perform a measurement or to apply the substance 10 or cream on the skin surface. Applying the cream is approximately equal to the actuation of the metering means 14.

The data stored in the memory unit 6 and/or the memory unit 16 may be transmitted to an external data receiver. The measuring apparatus 16 may be connected to a computer by means of a USB wire, for instance, and the data may then be transmitted to the external data receiver via the Internet.

As an alternative, the measuring apparatus 18 and/or the dispenser means 2 may comprise a conventional memory card such as a SD card, for instance. Data may be transferred onto this memory card via the processor unit 4 and/or the processor unit 28. The card may be inserted into the data reader device of a computer and the data may then also be transmitted to an external data receiver via the Internet.

In addition, the substance 10 may comprise a marker. After the substance 10 has been applied on the skin surface, the marker may be detected by a sensor of the sensor unit of the measuring apparatus 18. The marker cannot be resorbed by the skin and no interaction occurs between the marker and the chemical components of the substance 10. For instance, the marker may comprise particles of a size that prevent them from penetrating the skin. Moreover, the marker may be a fluorescent substance that can be detected by a sensor of the measuring apparatus 18 by means of fluorescence measurement.

In a method of the present invention which serves to measure changes in skin parameters of living beings on a skin surface to be examined as a function of the residence time o a substance, the skin parameters are measured on the untreated skin surface to be examined using the sensors 20 of the measuring apparatus 18 and the measured skin parameters are stored in the memory unit 16 and/or in the memory unit 6. The time of measurement can also be stored.

In a further step, the substance to be applied is identified. For instance, this may be done by inputting an identification code via the keyboard 22 of the measuring apparatus 18.

In the next step, a substance which is to be applied on the skin surface to be examined and which is contained in a container 12 of the dispenser means 2, is dispensed using the metering means 14 of the dispenser means 2 and is applied on the skin surface to be examined.

In the next step, the time of dispensing the substance 10 is stored. The time of dispensing the substance 10 is approximately equal to the application of the substance 10 on the skin surface. In addition, the quantity of the substance 10 dispensed can be detected and stored. The time of dispensing the substance 10 and, if the quantity of the substance 10 is detected, the quantity of the substance 10 dispensed are stored in the memory unit 16 and/or the memory unit 6, associated with each other and with the identification code.

In the next step, the skin parameters are measured again after one or a plurality of residence times using the sensors 20 of the measuring apparatus 18. The residence time is the time that has passed since the application of the substance 10.

In the next step, the time of the at least one new measurement of the skin parameters and the skin parameters measured are stored. The time of measurement and the measurement results are stored in the memory unit 16 and/or the memory unit 6, associated with each other and with the identification code.

Although the invention has been described and illustrated with reference to specific embodiments thereof, it is not intended that the invention be limited to those illustrative embodiments. Those skilled in that art will recognize that variations and modifications can be made without departing from the true scope of the invention as defined by the claims that follow. It is therefore intended to include within the invention all such variations and modifications as fall within the scope of the appended claims and equivalents thereof.

1. A system for measuring changes in skin parameters of living beings on a skin surface to be examined as a function of the residence time of a substance, comprising at least one measuring apparatus for measuring the skin parameters,
wherein
the measuring apparatus is coupled with at least one dispenser means which when operated dispenses a substance associated with this dispenser means and applicable on the skin surface to be examined, the measuring apparatus recording in a memory the time of actuation of the dispenser means and the time of the at least one measurement of the skin parameters for the detection of the residence time of the substance, as well as the skin parameters measured which are associated with the time of measurement.

2. The system of claim 1, wherein the dispenser means dispenses the substance in a predefined quantity.

3. The system of claim 1, wherein the substance is a pharmaceutical or cosmetic preparation.

4. The system of claim 1, wherein the measuring apparatus issues a reminder signal after a settable time interval or a plurality of settable time intervals.

5. The system of claim 1, wherein the measuring apparatus identifies the substance in the dispenser means by an identification code associated with the substance, and wherein the apparatus associates with this substance the measured values and times of measurement as well as the times of actuation of the dispenser means.

6. The system of claim 5, wherein the measuring apparatus transmits the measured values and times of measurement as well as the times of actuation of the dispenser means associated with a certain substance to at least one external data receiver.

7. The system of claim 1, wherein the measuring apparatus comprises a room climate sensor means.

8. The system of claim 1, wherein the measuring apparatus detects the skin elasticity and/or the color of the skin and/or the fat level of the skin and/or the sebum level of the skin and/or the skin humidity and/or the pH value of the skin.

9. The system of claim 1, wherein the measuring apparatus comprises at least one sensor means with which the presence of substances to be applied on the skin surface can be checked by measurement techniques, the sensor means detecting a marker in a substance in order to determine whether the substance marked with the marker is present on the skin surface to be examined.

10. The system of claim 9, wherein the marker does not interact with the chemical components of the substance and cannot be associated with any effect.

11. The system of claim 9, wherein the marker is not resorbable by the skin.

12. The system of claim 9, wherein the marker consists of particles, the particles being of such a size that they cannot penetrate into the skin.

13. The system of claim 9, wherein the marker is a fluorescent substance and the sensor means can detect the marker by means of fluorescence measurement.

14. A method for measuring changes in skin parameters of living beings on a skin surface to be examined as a function of the residence time of a substance by:
measuring the skin parameters on the untreated skin surface to be examined and storing the skin parameters measured,
dispensing a substance to be applied on the skin surface to be examined and applying the substance on the skin surface to be examined,
identifying the substance dispensed,
storing the time the substance was dispensed,
measuring again the skin parameters after one or a plurality of predetermined residence times, and
storing the time of the at least one new measurement of the skin parameters for a detection of the residence time of the substance.

15. The method of claim 14, wherein the quantity of substance dispensed is measured and stored.

16. The method of claim 14, wherein for checking the presence of substances to be applied on the skin surface by measurement techniques, a marker in a substance is detected in order to determine whether the substance marked with the marker is present on the skin surface to be examined.

17. The method of claim 14, wherein the marker does not interact with the chemical components of the substance (10) and cannot be associated with any effect.

18. The method of claim 14, wherein the marker is not resorbed by the skin.
